(19) United States
(12) Patent Application Publication
(10) Pub. No.: US 2025/0216759 A1
(43) Pub. Date: Jul. 3, 2025

(54) OTOSCOPE WITH DUAL FREQUENCY BANDS

(71) Applicant: GUANGZHOU LUXVISIONS INNOVATION TECHNOLOGY LIMITED, GUANGZHOU (CN)

(72) Inventor: Chih-Ju Lin, Guangzhou (CN)

(73) Assignee: GUANGZHOU LUXVISIONS INNOVATION TECHNOLOGY LIMITED, GUANGZHOU (CN)

(21) Appl. No.: 18/443,319

(22) Filed: Feb. 16, 2024

(30) Foreign Application Priority Data
Jan. 2, 2024 (CN) 202410003666.6

(51) Int. Cl. G03B 21/20

(52) U.S. Cl. CPC G03B 21/204; G03B 21/2013; G03B 21/2066

(57) ABSTRACT

An otoscope with dual frequency bands includes a plurality of first light sources, a plurality of second light sources and an optical sensing device. The first light sources and the second light sources emit light with a first frequency band and a second frequency band respectively, wherein at least a portion of the second frequency band does not overlap with the first frequency band. The optical sensing device includes a first sensing element and a second sensing element. The first sensing element is for sensing light with a third frequency band. The second sensing element is for sensing light with a fourth frequency band. The third frequency band includes the first frequency band, and the fourth frequency band includes the second frequency band.

### CROSS-REFERENCE TO RELATED APPLICATION

This application claims the priority benefit of China application serial no. 202410003666.6, filed on Jan. 2, 2024. The entirety of the above-mentioned patent application is hereby incorporated by reference herein and made a part of this specification.

### BACKGROUND

### Technical Field

This disclosure relates to an optical sensing device, particularly relates to an otoscope with dual frequency bands.

### Description of Related Art

When a foreign object enters the human ear, it might cause ear canal infection, otitis media, and hearing loss. Clinically, the fastest and most direct way is to check by inserting an otoscope into an external auditory canal for observation. In principle, it is possible to observe the conditions of the external auditory canal and the eardrum. In recent years, otoscopes have also been developed into digital otoscopes, which may record the real time image during observation, and a doctor may also keep an appropriate distance from patients.

However, the light sources of current otoscopes for capturing images are visible light, it causes that it's impossible to obtain information from middle ear (the subsections of the other side of the eardrum) clearly. Specifically, the most common disease of middle ear is otitis media with effusion (the middle ear fluid accumulation) caused by inflammation of middle ear (or otitis media). It caused water to be accumulated in the Eustachian tube and the middle ear cavity, which contains the tympanum and three ossicles. Water accumulation in the middle ear affects the vibration of the tympanum and the three ossicles, thereby affecting hearing sense and balance. The water accumulation is actually tissue fluid derived from inflammation. If the tissue fluid is yellowing interstitial fluid, it is still possible to be observed through the eardrum by a traditional otoscope. However, if the tissue fluid is too full in the middle ear, and the color of the tissue fluid is closed to be transparent, it is hard for a doctor to distinguish from whether there is water accumulation via the naked eyes or the current visible otoscope.

### SUMMARY

This disclosure provides an otoscope with dual frequency bands which may provide an optical testing within both visible light frequency band and infrared light frequency band simultaneously.

According to an embodiment of the disclosure, an otoscope is provided, including a sensing part. The sensing part includes a case body, a plurality of first light sources, a plurality of second light sources, and an optical sensing device. The case body includes an aperture and a frontal plane surrounding the aperture. The first light sources are disposed on the frontal plane for emitting a first light with a first frequency band. The second light sources are disposed on the frontal plane for emitting a second light with a second frequency band, wherein at least a portion of the second frequency band does not overlap with the first frequency band, and the first light and the second light is for illuminating a target to be detected to generate a light to be detected. The optical sensing device is disposed inside the case body, and includes a dichroic mirror, a first sensing element and a second sensing element. The light to be detected passes through the aperture and is separated by the dichroic mirror to form a third light and a fourth light respectively. The first sensing element is disposed on a path of the third light and for sensing a light with a third frequency band. The second sensing element is disposed on a path of the fourth light and for sensing a light with a fourth frequency band. The third frequency band includes the first frequency band, and the fourth frequency band includes the second frequency band.

Based on the above, other than illuminating the ear with visible light, the otoscope according to the embodiment of the disclosure also illuminates the ear with infrared light. Accordingly, for a material with a lower absorption coefficient in a visible light frequency band, or a material with a transparent form, such as water, it is possible to determine whether the water is accumulated in the ear by illuminating the ear with infrared light.

To make the aforementioned more comprehensible, several embodiments accompanied with drawings are described in detail as follows.

### BRIEF DESCRIPTION OF THE DRAWINGS

FIG. 1A is a curve diagram of absorption coefficient for water in near-infrared and short-wavelength infrared frequency band.

FIG. 1B and FIG. 1C are the visible light and infrared light pictures of a simulation of the water accumulation in the middle ear according to the embodiment of the disclosure.

FIG. 2A is a front and rear view of the otoscope according to the embodiment of the disclosure; FIG. 2B is a schematic diagram of a sensing part according to the embodiment of the disclosure.

FIG. 3A is the schematic diagram of the sensing part according to the embodiment of the disclosure; FIG. 3B is a schematic diagram of a frontal sensing part according to the embodiment of the disclosure.

FIG. 4 is a block diagram of the otoscope according to the embodiment of the disclosure.

### DESCRIPTION OF THE EMBODIMENTS

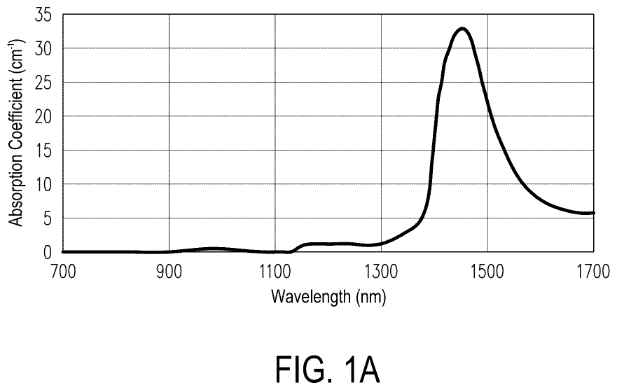

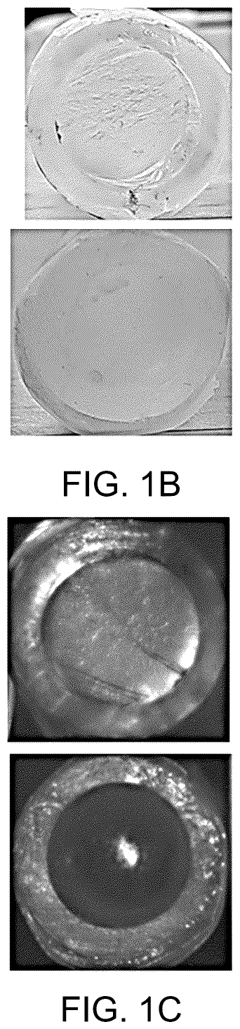

Referring to FIG. 1A, FIG. 1A illustrates a curve diagram of absorption coefficient for water in near-infrared and short-wavelength infrared frequency band. At approximately 1460 nm in the infrared light range, the absorption coefficient of water molecules has a local maximum value. Clinically, the fluid caused by otitis media with water accumulation is interstitial fluid, and the main component of interstitial fluid is water. In other words, if there is otitis media with water accumulation, the infrared light may be adopted to illuminate for performing clinical examination.

Referring to FIG. 1B and FIG. 1C for details. A silicone tube is for simulating the external auditory canal, a bio-membrane is for simulating the eardrum, and the bio-membrane is for sealing both ends of the silicone tube to form an enclosed space. The above device is then illuminated with the visible light or an infrared light source with a wavelength of 1460 nm, and obtain images with a visible light camera or an infrared light camera to generate images FIG. 1B and FIG. 1C. The top picture in FIG. 1B is an image obtained with the visible light camera when no water is placed in the enclosed space and the above device is illuminated with the visible light; the bottom picture in FIG. 1B is an image obtained with the visible light camera by filling the enclosed space with water and the above device is illuminated with the visible light; the top picture in FIG. 1C is an image obtained with the infrared light camera when no water is placed in the enclosed space and the above device is illuminated with infrared light in a wavelength of 1460 nm; the bottom picture in FIG. 1C is an image obtained with the infrared light camera by filling the enclosed space with water and the above device is illuminated with the visible light in a wavelength of 1460 nm. It is observable that the infrared light with a wavelength of 1460 nm and the infrared light camera may clearly distinguish whether there is water in the silicone tube, as shown in FIG. 1C. In contrast, since water is transparent in the visible light frequency band, it is impossible to correctly determine whether there is water in the silicone tube by illuminating water with the visible light and using the visible light camera, as shown in FIG. 1B.

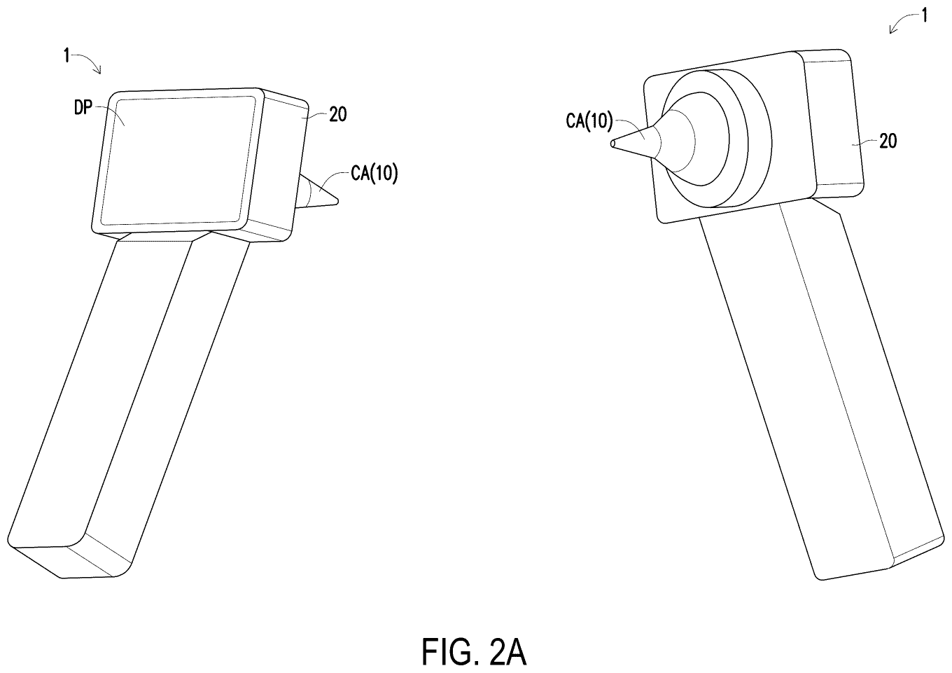

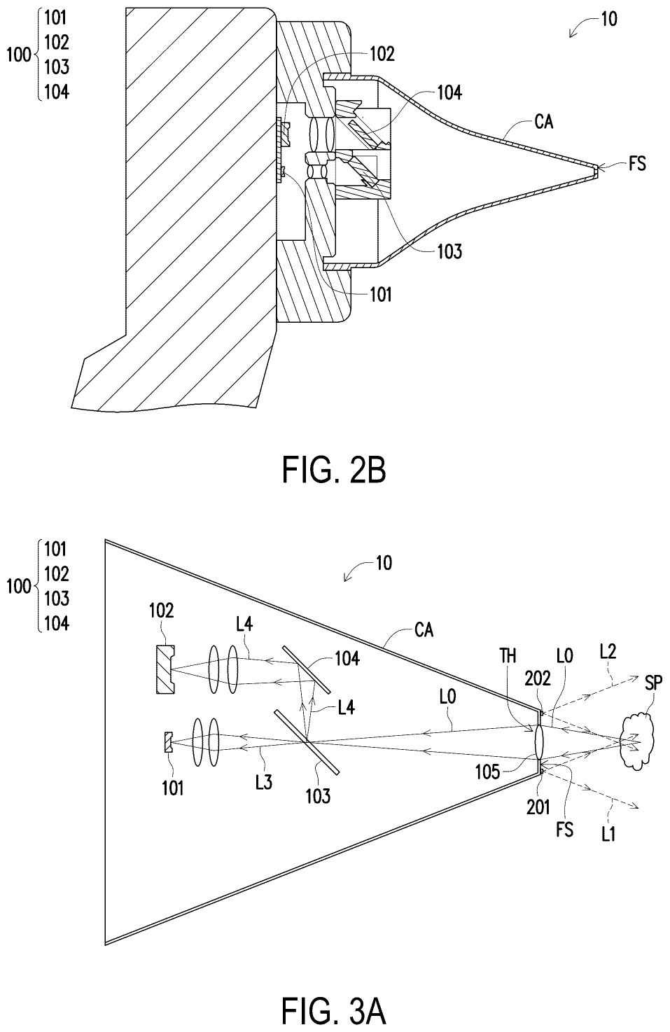

Referring to FIG. 2A, FIG. 2B, FIG. 3A, and FIG. 3B, FIG. 2A is a schematic diagram of an otoscope according to an embodiment of the disclosure; FIG. 2B and FIG. 3A are the schematic diagrams of a sensing part according to an embodiment of the disclosure; FIG. 3B is a schematic diagram of a frontal sensing part according to an embodiment of the disclosure.

An otoscope 1 includes a sensing part 10 and a control part 20. The sensing part 10 includes a case body CA, a plurality of first light sources 201, a plurality of second light sources 202 and an optical sensing device 100. The case body CA includes an aperture TH and a frontal plane FS surrounding the aperture TH. The first light sources 201 are disposed on the frontal plane FS for emitting a first light L1 with a first frequency band. The second light sources 202 are disposed on the frontal plane FS for emitting a second light L2 with a second frequency band, and the first light L1 and the second light L2 are for illuminating a target to be detected SP to generate a light to be detected L0 reflected and scattered from the target to be detected SP. The target to be detected SP may be, for example, a tissue in the ear, a foreign object in the ear, water in the ear, etc.

Since the length of the human external auditory canal is approximately 25 mm, the inner diameter is 4 to 8 mm. The maximum diameter (outer diameter) of the frontal plane FS may be more than 3 mm to facilitate placing the otoscope 1 into the external auditory canal for a certain distance to avoid interference from external light and to observe the conditions in the ear canal more clearly.

In the embodiment, the first light sources 201 may be an infrared LED, the first frequency band is from 1200 nm to 1600 nm. The second light sources 202 may be the infrared LED, the second frequency band is from 400 nm to 800 nm. That is to say, the second frequency band does not overlap with the first frequency band. But the invention is not limited to this, in some embodiments, the second frequency band may overlap partially with the first frequency band.

Specifically, other than illuminating the ear with the visible light LED, the otoscope 1 according to the embodiment also illuminates the ear with the infrared LED light. Accordingly, for a material with a lower absorption coefficient in a visible light frequency band, or a material with a transparent form, such as water, it is possible to determine whether the water is accumulated in the ear by illuminating the ear with infrared light. The infrared light may be Near-Infrared (NIR), Short-Wavelength Infrared (SWIR), Mid-Wavelength Infrared (MWIR), Long-Wavelength Infrared (LWIR) or Far-Infrared (FIR).

The optical sensing device 100 is disposed inside the case body CA and includes a dichroic mirror 103, a first sensing element 101 and a second sensing element 102. The wavelength boundary for light splitting of the dichroic mirror 103 falls within approximately 1000 nm. The first sensing element 101 may include indium gallium arsenide to sense light of 900 nm to 1700 nm frequency band (the third frequency band). The second sensing element 102 may be, for example, CCD or CMOS to sense light of 350 nm to 1100 nm frequency band (the fourth frequency band).

A lens 105 with diopter may be disposed at the aperture TH. The light to be detected L0 reflected and scattered from the target to be detected SP is refracted by the lens 105 when passing through the aperture TH, and then split by the dichroic mirror 103 so as to form a third light L3 passed through the dichroic mirror 103 and a fourth light L4 reflected by the dichroic mirror 103 respectively. The wavelength range of the third light L3 is greater than 1000 nm, and the wavelength range of the fourth light L4 is less than 1000 nm, so they may be sensed by the first sensing element 101 and the second sensing element 102 respectively.

The first sensing element 101 is disposed on a path of the third light L3, and one or a plurality of lens may be disposed between the dichroic mirror 103 and the first sensing element 101. The second sensing element 102 is disposed on a path of the fourth light L4, also a reflector 104 and one or a plurality of lens may be disposed between the dichroic mirror 103 and the second sensing element 102. The lens may include materials with a transmittance of at least 80% in the visible light and short-wavelength infrared, for example N-BK7, UV fused silica, N-SF11, CaF₂, and MgF₂. In addition, since an anti-reflective coating may be used to increase the transmittance of lenses to enhance the image signal, these lenses may be coated with coatings to reduce the occurrence of reflections. For example, a lens coated with the anti-reflective coating for 350 nm to 700 nm is disposed between the reflector 104 and the second sensing element 102. A lens coated with the anti-reflective coating for 1050 nm to 1700 nm is disposed between the dichroic mirror 103 and the first sensing element 101.

In some embodiments, a filter may further be disposed between the reflector 104 and the second sensing element 102 to filter out the band above 1000 nm to prevent the second sensing element 102 from generating noise. In some embodiments, a filter may further be disposed between the dichroic mirror 103 and the first sensing element 101 to filter out the band below 1000 nm to prevent the first sensing element 101 from generating noise.

In the embodiment, three first light sources 201 and three second light sources 202 are disposed on the frontal plane FS, but the disclosure is not limited to this. The number of the first light sources 201 may be greater than or equal to three, and the number of the second light sources 202 may be greater than or equal to three. If the number of the first light sources 201 and the number of the second light sources 202 is smaller than three, uniform illumination may not be provided and sensing accuracy may not be reduced. Furthermore, the number of first light sources 201 and the number of second light sources 202 may be the same or different. By disposing at least three first light sources 201 and at least three second light sources 202 on the frontal plane FS of the case body CA, with the distance between each first light source 201 being the same, and the distance between each second light sources 202 being the same, sufficient and uniform lighting is provided.

Note that since the sensing frequency band (900 nm to 1700 nm) of the first sensing element 101 covers the luminescence frequency band (1200 nm to 1600 nm) of the first light source 201, and the sensing frequency band (350 nm to 1100 nm) of the second sensing element 102 covers the luminescence frequency band (400 nm to 800 nm) of the second light source 202, the otoscope 1 is capable of completely sensing the frequency band of the light to be detected L0.

In some exemplary embodiments, the first light sources 201 have a local maximum luminous intensity in the wavelength range of 1400 nm to 1500 nm. For example, the LED has a maximum luminous intensity at 1460 nm, and the full width at half maximum may be approximately 100 nm. Accordingly, the characteristic of water molecules with a higher absorption coefficient at approximately 1460 nm may be used for improving sensing accuracy.

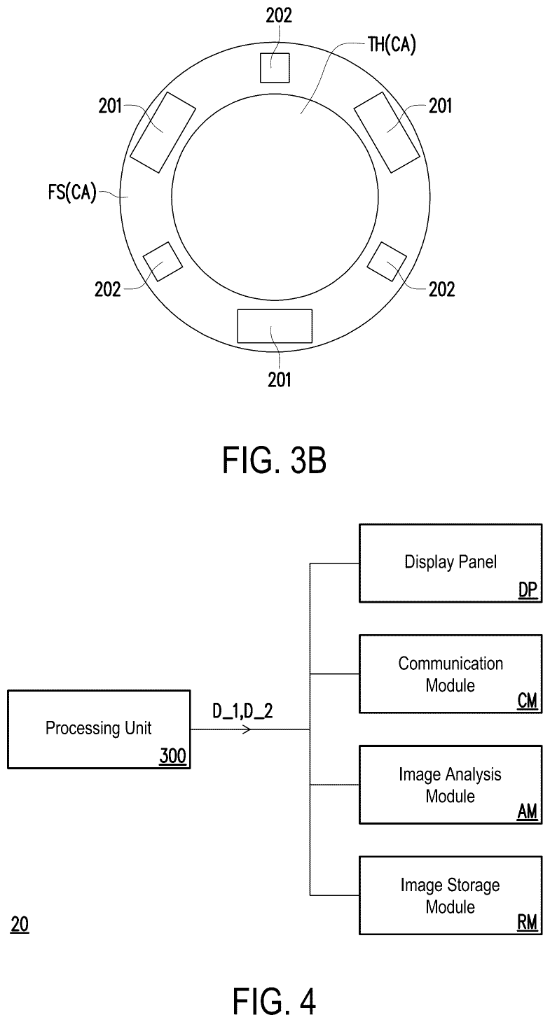

Referring to FIG. 2A and FIG. 4, the control part 20 of the otoscope 1 may include a processing unit 300. The processing unit 300 is electrically connected to the first sensing element 101 and the second sensing element 102 through a transmission interface to generate a first image data D_1 and a second image data D_2 respectively according to a sensing signal of the first sensing element 101 and a sensing signal of the second sensing element 102.

The control part 20 may further include a display panel DP to display an image corresponding to the first image data D_1 and an image corresponding to the second image data D_2.

The control part 20 may further include a communication module CM for transmitting the image corresponding to the first image data D_1 and the image corresponding to the second image data D_2.

The control unit 20 may further include an image analysis module AM for using AI to analyze the image corresponding to the first image data D_1 and the image corresponding to the second image data D_2 to learn whether there is foreign object intrusion or water accumulation in the ear.

The control part 20 may further include an image storage module RM for storing the image corresponding to the first image data D_1 and the image corresponding to the second image data D_2.

Based on the above, other than illuminating the ear with the visible light, the otoscope according to the embodiment of the disclosure also illuminates the ear with the infrared light. Accordingly, for a material with a lower absorption coefficient in a visible light frequency band, or a material with a transparent form, such as water, it is possible to determine whether the water is accumulated in the ear by illuminating the ear with infrared light.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. An otoscope comprises a sensing part, wherein the sensing part comprises:
a case body, comprising an aperture and a frontal plane surrounding the aperture;
a plurality of first light sources disposed on the frontal plane for emitting a first light with a first frequency band;
a plurality of second light sources disposed on the frontal plane for emitting a second light with a second frequency band, wherein at least a portion of the second frequency band does not overlap with the first frequency band, and the first light and the second light are for illuminating a target to be detected to generate a light to be detected; and
an optical sensing device disposed in the case body, and further comprising:
a dichroic mirror, wherein the light to be detected passes through the aperture and is separated by the dichroic mirror to form a third light and a fourth light respectively;
a first sensing element disposed on a path of the third light and for sensing a light with a third frequency band; and
a second sensing element disposed on a path of the fourth light and for sensing a light with a fourth frequency band,
wherein the third frequency band comprises the first frequency band, and the fourth frequency band comprises the second frequency band.

2. The otoscope according to claim 1, wherein the first frequency band is an infrared light frequency band and the second frequency band is a visible light frequency band.

3. The otoscope according to claim 1, wherein the plurality of first light sources have a local maximum luminous intensity in a wavelength ranging from 1400 nm to 1500 nm.

4. The otoscope according to claim 1, wherein the number of the plurality of first light sources is larger than or equal to three, and the number of the plurality of second light sources is larger than or equal to three.

5. The otoscope according to claim 1, wherein the first sensing element comprises indium gallium arsenide.

6. The otoscope according to claim 1, further comprising a control part, wherein the control part comprises a processing unit, the processing unit is electrically connected to the first sensing element and the second sensing element to generate a first image data and a second image data respectively.

7. The otoscope according to claim 6, wherein the control part further comprises a display panel for displaying a first image and a second image respectively corresponding to the first image data and the second image data.

8. The otoscope according to claim 7, wherein the control part further comprises an image analysis module for analyzing the first image and the second image.

9. The otoscope according to claim 7, wherein the control part further comprises an image storage module for storing the first image and the second image.

10. The otoscope according to claim 7, wherein the control part further comprises a communication module for transmitting the first image and the second image.
